(19) United States
(12) Patent Application Publication
(10) Pub. No.: US 2013/0203182 A1
(43) Pub. Date: Aug. 8, 2013

(54) METHOD FOR DECISION SUPPORT IN ALLERGY DIAGNOSIS

(71) Applicants: Karl Andersson, Uppsala (SE); Per Matsson, Uppsala (SE)

(72) Inventors: Karl Andersson, Uppsala (SE); Per Matsson, Uppsala (SE)

(73) Assignee: PHADIA AB, UPPSALA (SE)

(21) Appl. No.: 13/701,080

(22) PCT Filed: May 27, 2011
(86) PCT No.: PCT/SE2011/050664

§ 371 Date: Dec. 31, 2012

Related U.S. Application Data
(60) Provisional application No. 61/350,998, filed on Jun. 3, 2010.

(51) Int. Cl. G06F 19/00; G01N 33/68

(52) U.S. Cl. CPC G06F 19/34; G01N 33/68
USPC 436/501; 702/19

(57) ABSTRACT

A method of providing a clinical decision support in allergy diagnosis comprises the steps of: a) providing a body fluid sample from a patient, b) selecting a plurality of allergens to be tested for in the sample, c) determining for each allergen the concentration in the sample of at least one immunoglobulin directed against the allergen, d) transforming each determined immunoglobulin concentration to a clinical effect value on a normalized scale common to allergens in general, e) assigning to each allergen tested for, based on known cross-reactivity information for the allergen, an allergen specificity value, representing the degree of cross-reactivity for the allergen, and f) presenting determined clinical effect and allergen specificity values for each allergen, or a group or groups of the allergens. A computer-implemented method, a computer program product, and a patient information carrier device containing a diagnosis result are also disclosed.

### FIELD OF THE INVENTION

The present invention relates to the field of diagnosis of diseases, and more particularly to diagnosis of allergic conditions using a plurality of test-results for a patient. The invention also relates to a computer-implemented method for such diagnosis, a corresponding computer program product, and a patient information carrier device containing the result of the diagnosis.

### BACKGROUND OF THE INVENTION

Accurate diagnosis of diseases has always been one of the fundaments in health care. The process of making a diagnosis can take many forms, like symptom-, patient history- and test-based diagnosis. In test-based diagnosis, measured levels of biomarkers in body-fluids (e.g. IgE in blood is indicative of allergy, and sugar in the urine is indicative of diabetes) are used for pin-pointing the disease of the patient. Furthermore, physicians tend to combine symptom-based and test-based diagnosis in order to accurately state the underlying disease.

During the last few years, the availability of tests for biomarkers has increased immensely. It is now common that a panel of tests is performed and the result thereof, in combination with patient history, forms the basis for diagnosis. As long as the number of tests is less than around 20, this is acceptable for the average physician. However, when the physician has to make decisions based on 20-100 individual test results, the risk for misinterpretation and confusion increases drastically.

In order to improve the decisions made by the physician or decrease the number of misinterpretations and errors made, clinical decision support systems (CDSS) have been developed, designed to improve clinical decision making related to diagnostic or therapeutic processes of care. CDSSs address activities in many fields, such as the selection of drugs as described in Evans R. S., et al., “A computer-assisted management program for antibiotics and other anti-infective agents”, N Engl J Med 1998; 338:232-238, and the screening for latent tuberculosis infection as described in Steele, A. W., et al., “Using computerized clinical decision support for latent tuberculosis infection screening”, Am J Prey Med 2005; 28(3):281-4.

Within the field of diagnostics, a number of CDSSs have been disclosed to the public domain. Different support tools for interpretation of images are, for example, described in Harpole, L. H., et al., “Automated evidence-based critiquing of orders for abdominal radiographs: impact on utilization and appropriateness”, J Am Med Inform Assoc. 1997; 4:511-521; and in US 2005/102315 A1 entitled “CAD (computer-aided decision) support systems and methods”.

A CDSS for the interpretation of test results in a diagnostic situation is disclosed in EP1738168 A2, wherein a statistical method denoted “k-nearest-neighbors” (kNN) is applied to test results relating to autoimmune diseases. The kNN method compares the panel of novel test results (from a new patient) to the set of already diagnosed patients and classifies the new patient as having the same disease as the k existing results with most similarity to the new patient (wherein k is usually 3, 5 or any other small number). The kNN in EP1738168 relies completely on the collected data and cannot handle symptoms and other related information about the new patient.

Another CDSS is disclosed in WO 2009/099379 A1, which describes a clinical decision support based on test results for a plurality of tested biomarkers, wherein the biomarkers are hierarchically ordered. In one embodiment, scaling of measured entities into more interpretable units is performed, wherein probability-curves are used for improving the diagnostic quality of the statement if a particular patient is allergic or not.

Yet another CDSS for the interpretation of correlated ECG and heart-associated sound information is disclosed WO 2006/127022 A2. In this method, the use of two different types of information, i.e. ECG and heart-associated sound, are combined and presented in a common-display manner.

An example of a decision support system from a completely different field of science is disclosed in WO 97/44751, which relates to a method of presentation and interpretation of three-dimensional seismic data.

In allergy diagnostics, there is a trend to leave allergen extract based diagnostics and instead conduct diagnostic tests on allergen components (in the following frequently referred to as simply “components”). An extract-based test relies on immunological tests using a mixture of an allergy-causing entity, for example an extract of a particular species of grass. In a component based diagnostic procedure, on the other hand, the immunological test is made on relatively pure fractions of the extract, i.e. the components of the extract. Component based diagnostics improves the possibility to make more complete diagnostic statements, but instead requires deeper knowledge by the person interpreting the component based diagnostic test. There is presently, however, no CDSS available for such component based diagnosis of allergic conditions.

### SUMMARY OF THE INVENTION

It is an object of the invention to provide for clinical decision support in the diagnosis of allergy which simplifies the interpretation of multiple test results and which advantageously may be used in component based allergy diagnostics.

More particularly, it is an object of the invention to provide a method functioning as a CDSS that guides the physician to accurate allergy diagnosis when tens, hundreds, or even thousands of test results are available for a patient, and which presents the test result to the physician (and optionally also to the patient) in a simple and easily interpretable way.

This and other objects, which may appear from the description below, are at least partly achieved by means of a method for providing a clinical decision support, a computer-implemented method, a computer program product and a patient information carrier device according to the independent claims, embodiments thereof being defined by the dependent claims.

According to a basic concept of the present invention, each tested allergen (which may be an allergen extract or a pure component) is assigned (i) a first value based on the individual immunoglobulin concentration (typically IgE) in the patient's blood (or other body fluid) and which indicates the clinical effect, i.e. the clinical severity, of the immunoglobulin concentration, and (ii) a second value which is based on prior knowledge of cross-reactivity of each tested allergen, which two values each are normalized to a common scale, thereby providing a harmonized and easily interpretable decision support is to the physician. Thus, the present invention does not only provide information for assessing if a patient is allergic, but also how severe the allergy and how specific the allergy is (i.e. whether it is restricted to a single allergen source or is associated with several different allergen sources).

In a first aspect, the present invention provides a method of providing a clinical decision support in allergy diagnosis which comprises the steps of:

a) providing a body fluid sample from a patient,

b) selecting a plurality of allergens to be tested for in the sample, preferably blood,

c) determining for each allergen the concentration in the sample of at least one immunoglobulin directed against the allergen,

d) transforming each determined immunoglobulin concentration to a clinical effect value on a normalized scale common to allergens in general,

e) assigning to each allergen tested for, based on known cross-reactivity information for the allergen, an allergen specificity value, representing the degree of cross-reactivity for the allergen, and

f) presenting determined clinical effect and allergen specificity values for each allergen, or a group or groups of the allergens.

In a second aspect, the present invention provides a computer-implemented method of providing a clinical decision support in allergy diagnosis, which comprises the steps of:

(a) receiving test results from testing of a selection of allergens performed on a patient body fluid sample, wherein the test results are concentrations in the sample of at least one immunoglobulin directed against the allergens,

b) transforming each determined immunoglobulin concentration to a clinical effect value on a normalized scale common to allergens in general,

c) assigning to each allergen tested for, based on known cross-reactivity information for the allergen, an allergen specificity value, representing the degree of cross-reactivity for the allergen, and

d) presenting determined clinical effect and allergen specificity values for each allergen, or a group or groups of the allergens.

In a third aspect, the present invention provides a computer program product comprising instructions for causing a computer to perform the method steps of the computer-implemented method according to the second aspect above.

In a fourth aspect, the present invention provides an information carrier device comprising (i) a patient identifier code, (ii) a clinical effect value for at least one allergen, or allergen group, wherein the clinical effect value is on a normalized scale common to allergens in general, and (iii) an allergen specificity value representing the degree of cross-reactivity for the at least one allergen or allergen group.

Still other objectives, features, aspects and advantages of the present invention will appear from the following detailed description, from the attached dependent claims as well as from the drawings.

Embodiments of the invention will now be described in more detail with reference to the accompanying schematic drawings.

### BRIEF DESCRIPTION OF THE DRAWINGS

FIG. 1 is a schematic flow-chart of the method of the invention for providing a decision support in allergy diagnosis.

FIG. 2 illustrates an example of a presentation, intended for a physician, of test results transformed according to the method of the invention.

FIG. 3 illustrates an example of a presentation, intended for a patient, of test results transformed according to the method of the invention.

### DEFINITIONS

For the purpose of the following description, and for clarity, the following definitions are made:

A patient denotes a subject that may or may not have a disease. A patient is typically a human, but may be any living organism, in particular a mammal.

A body-fluid denotes a liquid in a living organism, including (but not limited to) blood, urine, tears, saliva, lymph, semen, faeces, etc.

A biomarker is a component in a body-fluid that can be detected or quantified using an analytical instrument. Biomarkers include, but are not limited to, proteins and metabolites.

Immunoglobulin denotes a complex group of proteins with antibody function, including but not limited to antibodies, e.g. IgA, IgD, IgE, IgG, and IgM, produced as a result of specific antigen-activated processes in the lymphoid organs. Antibodies of different specificities have different structures within the part of the molecule that binds antigen.

Allergy denotes an acquired, abnormal sensitivity to a foreign substance causing allergic symptoms.

An allergen denotes an entity capable of causing allergy. A test denotes the measurement of patient response to a particular allergen. An allergen is typically a mixture of two or more (allergen) components (usually proteins).

An (allergen) component denotes a reasonably pure allergen entity which alone can cause an allergic reaction. In typical cases, an allergen (e.g. birch pollen) comprises several different components, even though an allergen may consist of only one component. Thus, the term allergen includes all components. Furthermore, any single component can also be part of different allergens. A component is often a recombinant protein, i.e. prepared by genetic engineering.

An allergen source (also referred to as “host”) denotes the object/organism carrying or being associated with one or more particular allergen(s). A class of allergen sources denotes a group or family of allergen sources.

An epitope denotes a part of a macromolecule, such as an allergen, that is recognized by the immune system. An epitope is often a defined portion of a macromolecule, such as a few sequentially linked amino acids from a polypeptide or a protein, but can also be composed of different portions of a macromolecule which are brought in close vicinity in the 3D structure of the macromolecule. In general, an epitope is comparable to the size of a 300-3000 Da molecule.

Cross-reactivity relates to the ability of an allergen to elicit antibodies, specifically of IgE type, that will bind to a different allergen. This may be caused by an allergen component appearing in one or more different classes of allergens. Thus, a selected component may, for example, be present in an allergen X as well as, usually in a related variant form, in a different allergen Y. The allergen X is then said to be cross-reactive for the allergen Y, and vice versa. Cross-reactivity may also be due to an allergen component having several different epitopes (antigenic determinants), cross-reactivity occurring when non-identical components have an identical epitope or epitopes.

### DETAILED DESCRIPTION OF HE INVENTION

As mentioned above, a basic aspect of the present invention relates to a method for improving and simplifying the diagnosis of allergy.

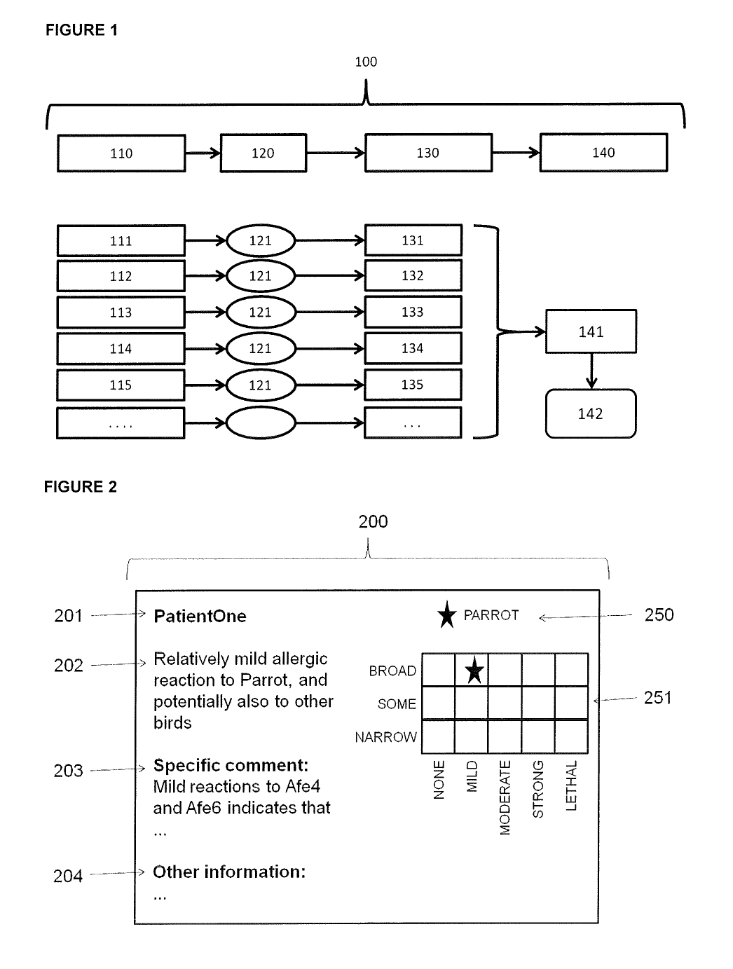

The method of the invention, which is designed to permit the generation of general and harmonized statements regarding an allergic condition of interest on the basis of a plurality of test results, will in the following be described in more detail with reference to FIG. 1.

From a simplified perspective, the method 100 comprises a first step 110 of selecting a plurality of allergens for which tests shall be conducted for a particular patient. The measurement of the patient reactivity to the selected allergens is then conducted in the second step 120. The test results are further processed in a third step 130 wherein the actual test reading (typically reported as a concentration unit or similar) is transformed into a clinical response scale. In the final fourth step 140 the transformed test values for all selected allergens and/or components are collected and presented to the physician. The method further includes the use of known cross-reactivity information for the allergen(s) in question in steps three 130 and four 140 to provide additional information to the physician. Such cross-reactivity information may, as mentioned above, for instance, be based on the knowledge that a particular allergen component may be present in several different allergen sources.

In more detail, the selected plurality of allergens for which tests shall be conducted for a particular patient in the first step 110 may consist of allergens 111-115, preferably components. The measurement of the patient reactivity to these selected allergens is then conducted in the second step 120, typically once for each allergen 121. A typical test of patient reactivity is the assessment of the concentration of the fraction of immunoglobulin type E (IgE) in the patient's blood (serum or plasma) which binds to the allergen, usually reported in the unit kU/L. The test results are further processed in a third step 130, wherein the each test reading is transformed into a clinical response scale.

It is well known that different allergens have different threshold values for apparent patient symptoms, i.e. a patient with a low value of a first allergen may be affected by allergy symptoms to a greater extent than a patient with a high value of a second allergen. In order to adjust for the different potency of the plethora of allergens, each allergen has an associated transform function 131-135 which takes the value as provided from the patient reactivity measurement and converts it into a general and harmonized scale of clinical effect or severity of allergic reaction, this harmonized scale being common to allergens in general.

The transformation functions 131-135 may also include cross-reactivity information which makes it possible to state the likelihood of allergic reactions to distantly related allergens, due to the fact that, as mentioned above, one and the same component may exist in several different allergens and/or an allergen or allergen component may share epitopes with a different component(s).

In the final fourth step 140 the transformed test values are compiled into a presentable format and then presented to the physician. Alternatively, or additionally, the cross-reactivity information may also be applied in this fourth step.

The selection of allergens in step 110 may be made with different objectives. For example, one possible objective is to select the fewest number of allergens that in concert can reveal an allergic reaction to the predominant allergies in the geographical area where the diagnosis is conducted. This objective essentially requires use of components, because the cross-reactivity pattern of components is known to a greater extent than to allergens in general.

Another example is where the objective is to refine the diagnosis of a patient with a known allergy. For example, if a patient knows that he or she is allergic to “Pollen” in a general sense, the selection of allergens could be made to accurately map the variety of “Pollen” from different plants from an allergy perspective. This would again essentially require use of components, because the cross-reactivity pattern of components is known to a greater extent than to allergens in general.

The measurement of the degree of patient reactivity in step 120 to a selected allergen can be conducted in different ways by a number of methods well known in the art.

Typically, however, an allergen of interest, which may be an extract or an allergen component, is immobilized on a carrier. When contacted with a patient's body fluid sample, usually a blood sample, specific IgE binds to the immobilized allergen. A labelled antibody against IgE is then added to form a complex which is detected via the labelled antibody.

In e.g. the commercial Immunocap™ system (Phadia AB, Uppsala, Sweden), the allergen is covalently coupled to a CNBr-activated cellulose derivative, and the anti IgE antibody is labelled by an enzyme which causes a fluorescent substance to be formed from an added substrate (developing agent). The fluorescence is then measured, and the higher the response value, the more specific IgE present in the sample. To evaluate the test results, the response for the patient samples are transformed to IgE concentrations by the use of a calibration curve.

The test result on the patient reactivity to the selected allergen may thus be a measurement of the concentration of a related indirect component, such as the concentration of IgE specific for the selected allergen. The obtained test result value per se is not necessarily indicative of the likelihood of allergy. Each allergen, and in particular each component, has a unique relationship between the test result and the clinical severity as partly determined on the inherent allergenicity of the respective allergen. It is therefore difficult for a physician to interpret a multitude of test results, because the test results are not necessarily comparable.

A transformation of test results into a generic scale of clinical severity therefore simplifies the interpretation of test results for the physician.

The transformation can potentially handle other relevant information, such as demographic data (age, race, domicile, etc), anamnesis, heredity factors, response pattern, genetic data, etc. For example, it is known that for some allergens, a positive test result of 10 kU/L is of moderate clinical severity for adults, but of high clinical severity for children.

The actual construction of the transformation, or scaling, can take many forms. For example, it can be a mathematical function that given a test result returns a clinical severity. Another possibility is to tabulate the clinical severity for a number of test results, and potentially use interpolation to achieve a clinical severity given a new test result. In a simple embodiment, the transformation is obtained from a curve (or graph) of clinical severity (or effect) versus IgE concentration.

The transformation as such can be designed to use any number of test results as input. Typically, 1-5 test results of similar (related) allergens are required for making an accurate generic statement of the allergy profile of the patient, but larger number of test results, such as at least 10, or at least 30, may be required in some cases.

A typical output of the transformation step 130 is a generic value of the clinical severity of each tested allergen or group of 2-20 (or even 20-2000) of allergens. The transformed values may, for example, be values between 1 and 5, where 1 denotes low clinical and 5 denotes high clinical severity. The term “value” in the present context is to be interpreted in a broad sense. The transformed values need therefore not be numbers, but can, for example, be a character scale (A-E) or a collection specific statements (“none”; “mild”; “moderate”; “strong”; “lethal”) or any other collection of numbers, words, symbols, colors, or pictures that can be apprehended as a scale.

Another typical output of the transformation step 130 is a generic value of the allergy specificity. Prior knowledge of the cross-reactivity pattern for a selected allergen or a group of 2-20 (or even 20-2000) similar allergens can be used to provide a more complete statement on the allergy status of the patient. In particular, use of cross-reactivity pattern is particularly useful when the tested allergens are components. In cases where mainly tests with a small degree of cross-reactivity are positive for a particular patient, the patient has a well-defined allergy profile. It is, for example, possible to tolerate nuts in general but with a specific exception for peanut. Such an allergy profile could be described as “narrow” or “specific”.

In cases where tests with a large degree of cross-reactivity are positive for a particular patient, the patient has a broad allergy profile. When such a patient responds positively to peanut, the patient would likely have allergic reactions to most types of nuts. Such an allergy profile could be described as “broad” or “non-specific”.

In summary, the use of prior knowledge on cross-reactivity can be used both for generating a more precise diagnosis for a particular patient, and for categorizing the specificity of the allergy of said patient as a “narrow responder” or a “general responder” in a predefined scale. Similarly as mentioned above for clinical severity, the scale of allergy specificity need not be a collection of statements, but can also be a numeric scale (such as values from 1 to 5), a character scale (A-E) or any other collection of numbers, words, symbols, colors, or pictures that can be apprehended as a scale.

In cases where a particular patient has been tested for allergens belonging to completely different classes of antigens (such as the two classes “Plants” and “Mammals with fur”), there will be two or more sets of diagnostic results, each set potentially comprising one value of clinical severity and one value of the specificity of the allergy. The plurality of sets of test results can be combined as indicated by step 141 in FIG. 1 into the same presentation of allergy status for a particular patient. This would enable an overview of the complete allergy diagnosis in one and the same report, and would potentially allow for comparisons of the clinical severity and the allergy specificity for this patient.

In the step of collecting and interpreting the transformed test results 140, there may be a need for different modes of presentation 142 after having conducted the above-mentioned combination 141 of the available test results. For example, one method of presentation could be available for physicians, which could indicate (i) potential additional allergies based on the allergy specificity and thus point out new tests that should be conducted for the patient, (ii) diet advise, (iii) statistic measures on the expected symptoms, (iv) potentially interfering medication for other (non-allergy related) diseases, and many other pieces of useful information. Another method of presentation could be available for the patient, which should contain much less medical information, instead focusing on simple facts and potential pro-active measures that the patient can act according to.

As is readily seen from the above, the present invention will improve the quality of diagnosis by transforming test results into generic, understandable scales. The transformation may, as mentioned previously, be applied to single test results or to groups of test results for similar or related allergens.

In step 140 in FIG. 1, the plethora of generic-scale values obtained for large set of test results may be combined, such as by use of secondary algorithms, into a set of information comprehendible for a physician or a patient.

In all cases, the present invention will, as already mentioned previously, benefit from test results obtained on pure components rather than on allergen extracts. The cross reactivity pattern is better known and the clinical severity transformation is more easily designed for components than for extracts from allergy-causing material. It is therefore preferred that more than 50%, more preferably at least 90%, and particularly all of the test results originate from components.

As already mentioned above, the present invention does not only provide the physician with support when making allergy diagnosis, but the generated information may also be presented to the patient, albeit in a simplified manner. Such patient-dedicated information is typically provided on an information carrier and includes: (i) patient information, e.g. in the form of a patient identifier code, and (ii) a summary of the related allergy information, e.g. in the form of a diagnosis code. The patient identifier information should preferably be anonymizable, and the diagnosis information should preferably be language-neutral.

In a simple embodiment, the information carrier is a card, suitably having a size in the range of about 10 mm*10 mm*1 mm to about 100 mm*150 mm*3 mm, preferably from about 25 mm*25 mm*1 mm to about 60 mm*90 mm*3 mm. The weight of such a card is preferably less than about 200 g, more preferably less than about 20 g, and most preferably less than about 7 g. The presentation of the information on the card may either be passive (for example printed) or active (for example by use of a digital display).

On the card, the patient name (or other identification) is presented together with information on the current allergy status, that is (i) clinical severity and (ii) allergy specificity (potentially rephrased in a simple wording). Optionally, additional information relevant to the allergy status of the patient may also be included, such as dietary information, particular materials or animals to avoid, and similar. This additional information should be presented in a short manner, using preferably less than 250 words, even more preferably about 10-50 words in total. Furthermore, the card may contain a unique identification code of the patient. This code may be used for retrieving additional information about the allergy of the patient through other sources, such as the internet or via mobile phones. The card may also contain an open access classification code which is a classification of the general allergy profile of the patient. By presenting the classification code in e.g. food-stores, the suitability of particular food-stuff may be presented to the patient.

Said codes (both the identification code and the classification code) may be stored on the card in different manners. The codes could be printed, stored in a magnetic strip, present as a radio-frequency identification (RFID) unit, embedded in micro-electronics in the card, just to mention a few examples.

The assessment of the suitability of particular food-stuff in a store can also be more or less automated. For instance, food-stuff articles typically have some type of international article number or coding such as an EAN-13 barcode defined by the standards organization GS1. The EAN-13 barcodes are used worldwide for marking products often sold at retail point of sale. By matching the international article number(s) per se being linked to information concerning substances that may cause an allergic reaction to a patient (or customer) with the allergy status of the client (or customer) by means of the present invention, it will be possible to avoid or at least identify articles containing substances that may cause an allergic reaction to this particular patient.

The matching of the interaction article number and the allergy profile code can be performed in any suitable way, more or less automated. As described above, the information carrier for instance a card can be designed so that information about the allergy of a patient can easily be matched against an article already in a food store.

Instead of a card, the information carrier and the matching can be provided by means of a mobile phone typically having an application program, often referred to as an “app”. The mobile phone may further be equipped with a camera and software for interpreting photos of barcodes, as well as internet connection. This would facilitate the matching process of the barcode labeled product classification and the allergy classification profile, and make it possible to provide additional information on the particular class of product through communication with a server through the internet. This would be particularly advantageous for warning patients about withdrawn products (food products are sometimes accidentally contaminated with e.g. peanut particles and are consequently withdrawn from the market) and informing patients on suitable alternatives to a particular product the patient is allergic to, to name two possible examples.

By means of the invention, it is possible to match allergy information relevant to the allergy status of a particular patient already in a food store.

Although the above-mentioned examples mainly describes food-related allergy, the same principle can be applied on other types of allergy. Products for washing and cleaning can contain potential allergens, some textile products like clothing are sometimes treated with chemicals that may cause allergic reactions, and jewelry may contain various metals to which individuals may be allergic. In all these cases, and in other similar cases, a product code matching with a patient allergy profile is adequate.

The method of the invention may be implemented by software run on an electrical data processing device, such as a computer. Such software may be provided to the computer on any suitable computer-readable medium, including a record medium, a read-only memory, or an electrical or optical signal which may be conveyed via electrical or optical cable or by radio or other means. The computer program product can comprise computer program sub-modules for performing one or more method steps. In other words, when the different sub-modules are run on a processor, one or more of the inventive method steps are performed. The sub-modules would generally be implemented in software, although implementations completely in firmware, hardware or combination thereof are also feasible.

The present invention will now be further illustrated by the following non-limiting Example.

### EXAMPLE

In this (hypothetical type) example the following is assumed: The diagnosis of allergy to Parrot can be made by testing the reactivity of a patient to four different components: Afe4, Afe6, Bua1, and Bua3. The Afe components are known to cross-react with other birds, while as the Bua components are more specific for Parrots. The patient reactivity test reports the concentration of IgE interacting with the respective component in the unit kU/L, and the test equipment can report values ranging from 0 to 100 kU/L. Each component has an individual transformation of the test result into the generic scale of clinical severity and allergy specificity, as indicated in Table 1. The scale of clinical severity has five levels: “none”; “mild”; “moderate”; “strong”; “lethal”. The scale of allergy specificity has three levels: “narrow”; “some”; “broad”. Afe4 further has one scale for children up to 10 years old, and a different scale for older patients.

TABLE 1
List of the hypothetical components and their scales of clinical severity
and allergy specificity, as used in Example 1.

| Component name and | | |
|---|---|---|
| patient age | Clinical severity | Allergy specificity |
| Afe4 & Age <=10 years | 0-5 kU/L = none | broad |
| | 5-15 kU/L = moderate | |
| | 15-100 kU/L = strong | |
| Afe4 & Age >10 years | 0-5 kU/L = none | broad |
| | 5-10 kU/L = mild | |
| | 10-30 kU/L = moderate | |
| | 30-100 kU/L = strong | |
| Afe6, all ages | 0-15 kU/L = none | some |
| | 15-55 kU/L = mild | |
| | 55-100 kU/L = moderate | |
| Bua1, all ages | 0-15 kU/L = none | narrow |
| | 15-35 kU/L = mild | |
| | 35-100 kU/L = moderate | |
| Bua3, all ages | 0-25 kU/L = none | some |
| | 25-35 kU/L = mild | |
| | 35-45 kU/L = moderate | |
| | 45-100 kU/L = strong | |

Three patients (PatientOne, PatientTwo, and PatientThree) were tested for response to the four components Afe4, Afe6, Bua1, and Bua3. The test results (as expressed in kU/L) and the corresponding transformed generic values of clinical severity and allergy specificity are shown in Table 2.

TABLE 2
List of patient response as test results and as transformed generic
values in terms of clinical severity and allergy specificity.

| | | Test results (kU/L) | | | | Transformed results (severity; specificity) | | | |
|---|---|---|---|---|---|---|---|---|---|
| | Age | Afe4 | Afe6 | Bua1 | Bua3 | Afe4 | Afe6 | Bua1 | Bua3 |
| PatientOne | 13 | 7 | 17 | 3 | 27 | mild; | mild; | none; | mild; |
| | | | | | | broad | some | narrow | some |
| PatientTwo | 5 | 7 | 25 | 18 | 49 | Moderate; | mild; | mild; | strong; |
| | | | | | | broad | some | narrow | some |
| PatientThree | 24 | 2 | 4 | 78 | 40 | none; | none; | moderate; | moderate; |
| | | | | | | broad | broad | narrow | some |

The transformed test results for PatientOne indicates that PatientOne has a relatively mild allergic reaction to Parrot, and potentially also to other birds. This can be concluded because the highest clinical severity rating obtained was “mild” and the broader Afe components scored positive (Afe4=mild, Afe6=mild).

PatientTwo has a strong allergic reaction to Parrot, and most probably a moderate to strong reaction to other birds. This conclusion is supported by the strong reaction to Bua3, which has only “some” allergy specificity. The result for Afe4 was moderate, and is the main contributor to the allergy specificity statement.

PatientThree has a moderate reaction to Parrot, and will probably react in a mild fashion to other birds. The Bua1 and Bua3 components were the only ones resulting in positive values, and the allergy specificity of Bua1 and Bua3 is “narrow” and “some”, respectively.

The transformed test results can be presented in different manners depending on the recipient (or user) thereof. When presenting the results to a physician, there may be included images, graphs simplified messages, and the like for the immediate summary of the patient allergy response, and detailed information may be provided in the same report. An exemplary way of providing a result presentation for PatientOne is schematically shown in FIG. 2.

A report sheet 200 which contains a patient identification 201 and a brief written statement 202 which summarizes the allergic status of said patient. There is also a more specific description 203 of the underlying reasons for the summary statement and further a section with other related information 204, which could include e.g. dietary recommendations, medication recommendations, and similar related information. It is further advantageous to include on sheet 200 an alternative format of the summary information 202, preferably as a graph or a table 251 wherein the allergy reactions of said patient are plotted together with an accompanying legend 250.

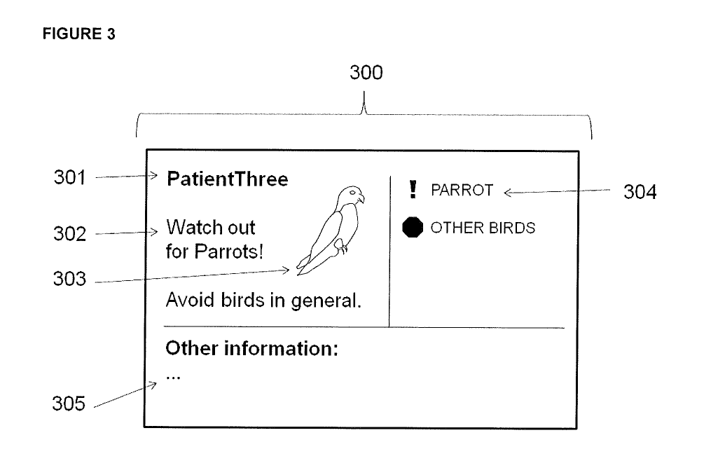

Likewise, when presenting the results to the patient, images, graphs, simplified messages, and the like for the immediate summary of the patient allergy response may also be included, but in this case the amount of detailed information should preferably be limited. One exemplary way of providing a result presentation of PatientThree is schematically shown in FIG. 3. A small report sheet 300 contains a patient identification 301 and a very brief written statement 302, potentially together with a small image 303 which summarizes which allergen sources the patient should avoid. It is further advantageous to include an alternative method of providing the summary information 304, potentially by use of symbols which already have a meaning for the patient. For patients in Europe, for example, two such examples may be the exclamation mark and the octagon (c.f. the stop sign for car drivers). Similar relationships with symbols exist in all parts and cultures in the world. Other useful information like the telephone number to the clinic may be provided in a separate field 305.

The invention has mainly been described above with reference to a few embodiments thereof. However, as is readily appreciated by a person skilled in the art, other embodiments than those disclosed above are equally possible within the scope and spirit of the invention, which is defined and limited by the appended claims only. For instance, instead of allergic reaction to parrot, the invention is also applicable to any other allergy-causing entity such as peanut.

1-13. (canceled)

14. A method of providing a clinical decision support in allergy diagnosis, comprising the steps of:
a) providing a body fluid sample from a patient,
b) selecting a plurality of allergens to be tested for in the sample,
c) determining for each allergen the concentration in the sample of at least one immunoglobulin directed against the allergen,
d) transforming each determined immunoglobulin concentration to a clinical effect value on a normalized scale common to allergens in general,
e) assigning to each allergen tested for, based on known cross-reactivity information for the allergen, an allergen specificity value, representing the degree of cross-reactivity for the allergen, and
f) presenting determined clinical effect and allergen specificity values for each allergen, or a group or groups of the allergens.

15. The method according to claim 14, wherein in step d) in claim 14, the immunoglobulin concentrations are transformed to a single clinical effect value for a group of related allergens.

16. The method according to claim 14, wherein at least one allergen is an allergen component.

17. The method according to claim 14, wherein at least 50%, preferably at least 90% of the allergens are allergen components.

18. The method according to claim 14, wherein at least ten allergens are tested for.

19. The method according to claim 14, wherein step b) in claim 14 comprises selecting one or more predetermined groups of allergens.

20. The method according to claim 14, wherein the at least one immunoglobulin comprises IgE.

21. The method according to claim 14, wherein said allergen components comprise recombinant allergens.

22. The method according to claim 14, wherein determined clinical effect and allergen specificity values for at least one allergen or group of allergens are provided on a patient information carrier device.

23. A computer-implemented method of providing a clinical decision support in allergy diagnosis, comprising the steps of:
a) receiving test results from testing of a selection of allergens performed on a patient body fluid sample, wherein the test results are concentrations in the sample of at least one immunoglobulin directed against the allergens,
b) transforming each determined immunoglobulin concentration to a clinical effect value on a normalized scale common to allergens in general,
c) assigning to each allergen tested for, based on known cross-reactivity information for the allergen, an allergen specificity value, representing the degree of cross-reactivity for the allergen, and
d) presenting determined clinical effect and allergen specificity values for each allergen, or a group or groups of the allergens.

24. A computer program product comprising instructions for causing a computer to perform the method steps of claim 23 when run in a processor.

25. An information carrier device comprising:
(i) a patient identifier code,
(ii) a clinical effect value for at least one allergen, or allergen group, wherein the clinical effect value is on a normalized scale common to allergens in general, and
(iii) an allergen specificity value representing the degree of cross-reactivity for the at least one allergen or allergen group.

26. The information carrier device according to claim 25, wherein the clinical effect value or values and the allergen specificity value or values have been obtained by the method according to claim 14.
